(19) United States
(12) Patent Application Publication
(10) Pub. No.: US 2011/0230954 A1
(43) Pub. Date: Sep. 22, 2011

(54) STENT DEVICE HAVING FOCAL ELEVATING ELEMENTS FOR MINIMAL SURFACE AREA CONTACT WITH LUMEN WALLS

(76) Inventors: Peter Schneider, Honolulu, HI (US); Robert Giasolli, Honolulu, HI (US)

(21) Appl. No.: 13/118,388

(22) Filed: May 28, 2011

Related U.S. Application Data
(63) Continuation-in-part of application No. 12/790,819, filed on May 29, 2010.
Application No. 12/790,819 is a continuation-in-part of application No. 12/483,193, filed on Jun. 11, 2009.
(60) Provisional application No. 61/349,836, filed on May 29, 2010.

(51) Int. Cl. A61F 2/82

(52) U.S. Cl. 623/1.15

(57) ABSTRACT

An improved stent device has a body structure in tubular form sized to an organ lumen in which it is to be used and made of a wire mesh or cage structure of interwoven or interdigitated strut sections, and a plurality of focal elevating elements of relatively small point-like size or vector-like edge in an array over outer surfaces of the wire mesh or strut sections of the stent body structure. The focal elevating elements elevate the wire mesh or strut sections away from and minimize surface area contact with the organ lumen walls. They also reduce contact pressure in regions neighboring the focal elevating elements in order to minimize trauma due to contact or movement against the organ lumen walls. A preferred use for the stent device is in a blood vessel lumen, particularly to retain plaque dissection after balloon angioplasty.

This U.S. Patent Application claims the priority filing date of U.S. Provisional Application 61/349,836 filed on May 29, 2010, and is a continuation-in-part of co-pending U.S. patent application 12/790,819 filed May 29, 2010, which was a continuation-in-part of co-pending U.S. patent application 12/483,193, filed Jun. 11, 2009, of the same inventors.

### TECHNICAL FIELD

This invention relates to an improved stent device for use in the body, and particularly a stent device that makes minimal surface area contact with lumen walls.

### BACKGROUND OF INVENTION

A stent may be placed during routine medical practice within a tubular body structure to restore or support its structural integrity. It can provide a structural scaffold within arteries, veins, bile ducts, pancreatic ducts, ureters, trachea, bronchus, ear canal, eye canal, sinuses, intestinal tracts, and other structures. Existing stent designs place an elongated scaffold structure of foreign material in contact with the surface of an organ lumen, which can induce a variety of macroscopic and microscopic responses from host tissues resulting in inflammation, scar formation, thrombus formation, and/or late failure due to tissue in-growth. A common problem is re-stenosis or accumulated blockage of the stent and its consequent failure to perform its intended function. Structure-specific properties of stents have also been associated with increased biological reactivity. There is also an increased risk of microfriction if there is any movement of the organ in which the lumen is located, and a tendency for the stent device to fracture over time.

An example of conventional stent usage is for clinical management of plaque dissection in blood vessels after balloon angioplasty treatment for an atherosclerotic condition. As illustrated in FIG. 1, a stent is typically a structure in a tubular form having a diameter that is sized to the diameter of the blood vessel and commonly made of a wire mesh or cage structure of interwoven or interdigitated strut sections. It is placed in the blood vessel lumen to hold any plaque dissection flaps against the blood vessel wall. As stents are made of metal alloys or other foreign materials, they are subject to recurrent stenosis of plaque accumulation in the treated blood vessel. Depending on the location and the size of the blood vessel, in-growth of intimal hyperplastic tissue from the vessel wall in between struts or through openings in the stent may occur and cause failure of the vascular reconstruction by narrowing or occlusion of the stent. This reaction of the blood vessel to the presence of the stent is likely due to the extensive use of foreign material and blood vessel surface contact.

### SUMMARY OF INVENTION

In accordance with the present invention, an improved stent device comprises:

a stent body structure in a tubular form having a length and an outer diameter sized to dimensions of an organ lumen in which it is to be used, and being made of a wire mesh or cage structure of interwoven or interdigitated strut sections;

a plurality of focal elevating elements of relatively small point-like size or vector-like edge arranged in an array at spaced locations over outer surfaces of the wire mesh or strut sections of said stent body structure, said focal elevating elements having a height which results in an elevated outer diameter greater than the outer diameter of the wire mesh or strut sections of said stent body structure, and having a total contact surface area for contact with walls of the organ lumen that is a fraction of a total contact surface area of the outer surfaces of the wire mesh or strut sections of said stent body structure, and being configured to apply point pressure to the organ lumen walls so as to elevate adjacent outer surfaces of the wire mesh or strut sections of said stent body structure away from contact with the organ lumen walls.

In preferred embodiments, the focal elevating elements have regionally lifted sections that are configured to reduce pressure in regions neighboring the focal elevating elements in order to minimize trauma due to contact or movement between the device and the organ lumen walls.

A stent device may be provided or enhanced with focal elevating elements on the annular periphery of the device. The focal elevating elements are distinguished from the anchors and barbs generally having greater plaque or arterial wall penetration to anchor or stabilize the stent in the blood vessel.

The focal elevating elements may or may not penetrate but still offer regional strut elevation and are preferably placed at apices and at bridges of struts or periodically either perpendicular, parallel, or at some angle to the strut lengths. For both anchors and focal elevating elements the size of the interface between the stent and the arterial wall is preferably equal to or shorter than the strut width in at least one direction. The focal elevating elements can be similar to anchors but either do not penetrate or penetrate the tissue only slightly, thereby minimizing the amount of material surface area in contact with the plaque, and offer a set of relief sections for the outward pressure of the stent device adjacent to the focal elevating elements, thereby minimizing the friction generated at the blood vessel wall.

The focal elevating elements can be formed and configured on the annular periphery of the stent device in a similar manner as described for the previous plaque tack device embodiments and can include the raised contact sections in addition to anchors or sharp points. The contact sections can provide improved stenting characteristics in that they increase the outward forces at the contact sections by compressing the plaque at the contact regions and decrease the outward force at the sections neighboring the focal elevating element. This offers regional pressure relief in some sections and increase pressure at the bumps or sharp points collectively offering a reduction in trauma and cellular response of the blood vessel wall.

Because the stent device is held in place by its own pressure exerted on the blood vessel surface, it is susceptible to friction, i.e., slight movement between the device and the vessel surface. Every time the organ moves (e.g., the leg during ambulation), the artery moves. It can be inferred that when the artery moves the working device sitting within the artery also moves but not necessarily every point of contact moves in synch with each other. Whenever there is even a small mismatch between the artery and the device the system rubs against each other promoting cellular reaction and device failure. It has been deduced from experimental data that this rubbing irritates the endothelium causing an inflammatory response.

In the present invention, strategically placed focal elevating elements (FEEs) are implemented to reduce the overall regional friction load (thought to be a source of inflammation, cellular proliferation, and the healing response that leads to re-stenosis) of the area being held open. These raised sections produced by the FEEs limit the histological response of the tissue and the fatigue of the device by limiting the contact between the device and the tissue. Independent of the volume of contact, the stent devices smooth the lumen wall, and allow more natural vessel movement. It is this micro-movement that increases the cellular response of the blood vessel surface to the foreign device.

The focal elevating elements are designed to reduce effective metal interface (EMI) by minimizing the overall material contact with the blood vessel surface. The focal elevating element (FEE) is preferably configured as a narrow, lifted feature with enough height to lift adjacent strut sections of the stent device off from contact with the arterial wall in order to reduce the surface area of foreign material in contact with the arterial wall. Reducing the contact burden is of particular value when the strut members are connecting circumferential rings or circumferentially oriented strut bands. Strut sections in contact with the blood vessel walls can produce microfriction when they move or rub against the blood vessel walls. By reducing the foreign material contact area against the blood vessel wall, the tendency for production of microfriction contact is reduced. Preferably, the EMI for the improved stent device is a small fraction of the EMI of a stent without focal elevating elements.

The focal elevating elements are configured to result in an outer diameter that is slightly greater than the outer diameter of the stent body structure. Preferably, the percentage difference in outer diameters is in a range of 10%-0.1%, and more preferably in the range of 4%-0.5%, and in one embodiment is approximately 1.5%.

The focal elevating elements are configured to have a ratio of height of the focal elevating elements compared to thickness of the strut sections of the stent body structure preferably in a range of 0.05 to 3, and most preferably in the range of 0.2 to 0.7. The height of the focal elevating elements is in a preferred range of 0.02 mm to 4 mm, and most preferably in the range of 0.05 mm to 0.2 mm.

The focal elevating elements may be formed on strut sections of a stent body with a cylindrical, pyramidal, tent, or dome shape. They may be placed along a length of a strut section or at an apex of a strut section. They may be bent from a portion of a strut length to raise it in height above the surface of neighboring strut length, an apex of a strut section bent upward, or an apex of a strut section folded upward.

The focal elevating elements are formed by laser-cutting or etching a cylindrical blank that has two outer-diameter levels of structure: a cylindrical blank for the stent body structure having a lesser outer diameter; and annular concentric rings superimposed on the stent body structure having a greater outer diameter, which are laser-cut or etched to form spaced arrays of focal elevating elements on the stent body structure.

In another preferred embodiment, the focal elevating elements (FEE) are small fluke-like segments of a given length formed on strut sections of the stent body structure. Preferably, a ratio of length of the FEE segments measured along an axial distance of the FEE to strut thickness is in a preferred range of 0.2 to 10. Preferably, the fluke-like segments are oriented to extend either along a longitudinal or perpendicular axis of the stent device or at an angle/angles that offer cellular contact and microfriction relief.

Properly oriented and symmetrically positioned focal elevating elements can provide foci for expansion forces. As the device exerts outward forces and the artery exerts inward forces, the focal elevating elements can be positioned at strategically located positions reducing the outward pressure of strut sections neighboring the focal elevating elements.

Both anchors and focal elevating elements can offer strategic advantages that include: the reduction in pressure burden across the stent struts by reducing the contact area and translating the outward forces to the anchors and focal elevating elements, minimizing surface contact which offers a reduction in the tendency of frictional loading driven by micro movement between the arterial wall and the stent strut, and the stabilization of anchoring the stent where the anchor or focal elevating element penetrates the vessel wall a fraction of the features height.

In other preferred embodiments, the focal elevating elements are formed on bridge portions joining adjacent annular rows of strut sections, or formed on adjoining apices of adjacent annular rows of strut sections. They may also be formed as a twisted loop of wire at an intersection of wire mesh sections of the stent body structure. They may also be formed as a fluke-like segment straddling an intersection of crossing strut sections of the stent body structure.

A preferred environment of use for the improved stent device is in a blood vessel lumen, particularly to retain plaque dissection in a blood vessel lumen after balloon angioplasty. It can also be used in arteries, veins, bile ducts, pancreatic ducts, ureters, trachea, bronchus, ear canal, eye canal, sinuses, intestinal tracts, and other tubular organ structures.

Other objects, features, and advantages of the present invention will be explained in the following detailed description of the invention having reference to the appended drawings.

### BRIEF DESCRIPTION OF DRAWINGS

FIG. 1 illustrates the use of a conventional stent installed for clinical management of plaque dissection in a blood vessel after balloon angioplasty (prior art).

FIG. 2 is a schematic diagram illustrating the use of focal elevating elements in a stent device.

FIG. 3 is a schematic diagram illustrating the resulting forces from use of a stent device with focal elevating elements (FEEs) for clinical management of plaque dissection in a blood vessel.

FIG. 4 illustrates a perspective view of a series of focal elevating elements (FEEs) spaced along the length of a strut section of a stent device.

FIG. 5 illustrates a detailed view of a cylindrically shaped FEE placed at the apex of a strut section of the stent device.

FIG. 6 illustrates a perspective view of a FEE formed as a pyramidal element at the apex of a strut section.

FIG. 7 illustrates a perspective view of a FEE formed as a dome element at the s apex of a strut section.

FIG. 8 illustrates a perspective view of a FEE formed by bending a portion of a strut length to raise it in height above the surface of the neighboring strut length.

FIG. 9 illustrates a perspective view of a FEE formed by bending the apex of a strut section upward.

FIG. 10 illustrates a perspective view of a FEE formed by folding the apex of a strut section upward.

FIG. 11 illustrates a perspective view of focal elevating elements (FEEs) laser-cut or etched on the surface of a strut section of a stent device.

FIG. 12 illustrates a perspective view of a FEE laser-cut or etched as a bar element on the bend of a strut section.

FIG. 13 illustrates a perspective view of a FEE laser-cut or etched as a fluke-like segment on a horizontal bend in the length of a strut section.

FIG. 14 illustrates a perspective view of a FEE laser-cut or etched as a fluke-like segment straddling a bend of a strut section.

FIG. 15 shows a sequence of progressively enlarged views of a stent mesh structure with rows of strut sections joined by bridge portions between adjacent strut sections.

FIG. 16 illustrates a cylindrical blank having two outer-diameter levels of structure for forming focal elevating elements by laser-cutting or etching.

FIG. 17 illustrates the resulting stent structure formed with focal elevating elements by laser-cutting or etching the cylindrical blank in FIG. 16.

FIG. 18 illustrates a perspective view of a FEE formed by twisting a loop at an intersection of wires of a stent mesh structure.

FIG. 19 illustrates a perspective view of a FEE laser-cut or etched as a fluke-like segment straddling the intersection of two crossing strut sections.

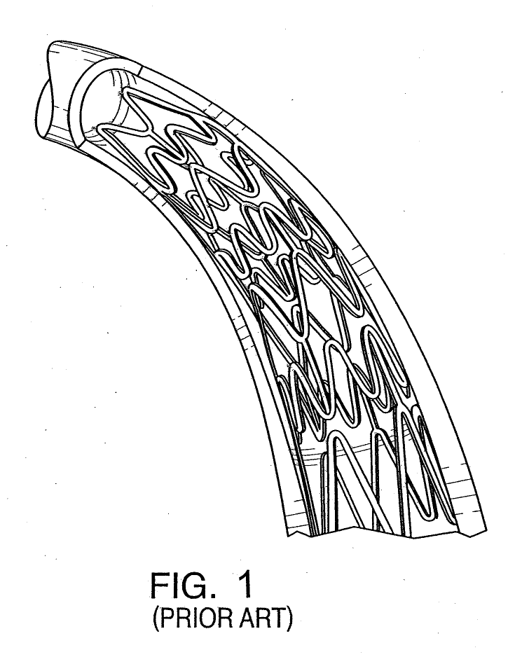

### DETAILED DESCRIPTION OF INVENTION

In the present disclosure, a stent may be improved in design and performance in the human body by the use of focal elevating elements on the outer surface of the device to minimize surface area contact of the stent material with the organ lumen walls. The focal elevating elements operate to elevate adjacent sections (struts) of the stent away from the organ lumen walls to minimize friction generated at contact areas. The focal elevating elements increase outward pressure at contact points with the organ lumen walls to maintain them in stable contact, while elevating the adjacent sections of the stent out of contact with the organ lumen walls. The focal elevating elements (FEEs) are strategically placed to reduce the overall regional friction load (thought to be a source of inflammation, cellular proliferation, and the healing response that leads to re-stenosis) of the area being held open. These elevated contact points of the FEEs are believed to limit the histological response of the tissue and the fatigue of the device by limiting contact between the device and the tissue walls which experience external forces during normal lower extremity movement during the course of daily activities.

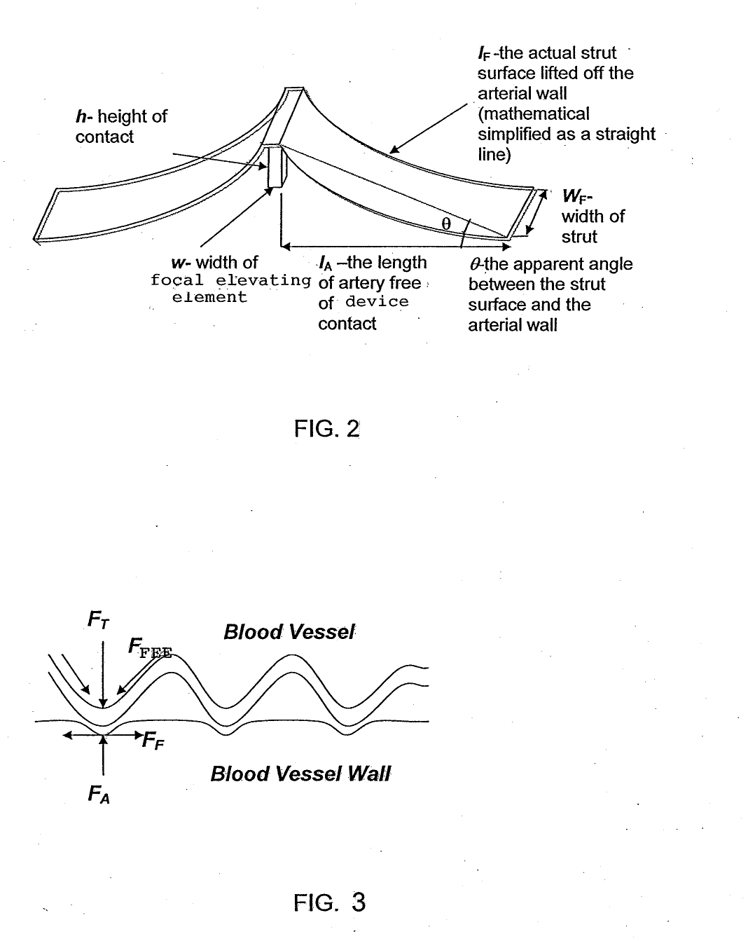

Referring to FIG. 2, a schematic diagram illustrates the preferred design assumptions for the use of focal elevating elements on a stent. In the figure, h refers to the height of the focal elevating element that is measured from the outer diameter of the stent radially towards the blood vessel. If the focal elevating elements are designed to be anchored into the artery walls, the penetration depth is not included in this height calculation. The variable w refers to the width of the focal elevating element (at its base), and l_{F} refers to the adjacent strut surface lifted off the arterial wall (mathematically simplified as a straight line). The struts adjacent to the focal elevating element may be fabricated with shape memory materials or designed as a compression wave providing compensation for lumen diameter variations. The strut forces adjacent to the focal elevating elements produce an outward bowing of the struts produced by the forces of the struts wanting to expand until they are in contact with the blood vessel wall. l_{A} refers to the length of arterial wall that is kept out of contact with any adjacent strut structure by the focal elevating element. The height h of a FEE can vary while the angle

[symbol]

formed by the intersection of the length l_{A} of artery free of contact with the hypotenuse line from the strut surface to the arterial wall is preferably kept constant.

In an example of use of the improved stent device with focal elevating elements in a blood vessel, FIG. 3 illustrates the resulting forces between the stent's focal elevating elements and the arterial wall. F_{T} is the circumferential force exerted by the stent device against the arterial walls force, F_{A}. F_{FEE} is an additive circumferential force at the focal elevating element generated by the design and material choice and F_{F} is the frictional force of the artery generated when the artery changes its orientation or shape due to body forces. Every time a body party moves, the blood vessels move slightly as well. The focal elevating elements can be strategically positioned to reduce local friction loading which may cause inflammation, cellular proliferation, or bodily response that leads to re-stenosis.

The focal elevating elements (FEEs) may be formed as cylindrical, rectangular, spherical, conical, tear dropped, pyramidal, or inclined elements on the periphery of strut sections of a stent device. They can be formed by bending or stamping a section of the stent structure, by an additive process (such as by welding or annealing on a peripheral surface), by a subtractive process (such as by grinding or etching away surrounding material) so that the FEE element is higher than the surrounding surface, or by modifying small sections of the peripheral surface to be higher than the surrounding surface before or after sheet or tube cutting. For example, one method of modification of small sections of a mesh stent structure is by knotting, twisting, bending or weaving small sections of the mesh to produce raised elements from the mesh surface which are the interface with the artery wall. Examples of preferred designs for the focal elevating elements on strut sections of a stent body structure are shown in the following figures.

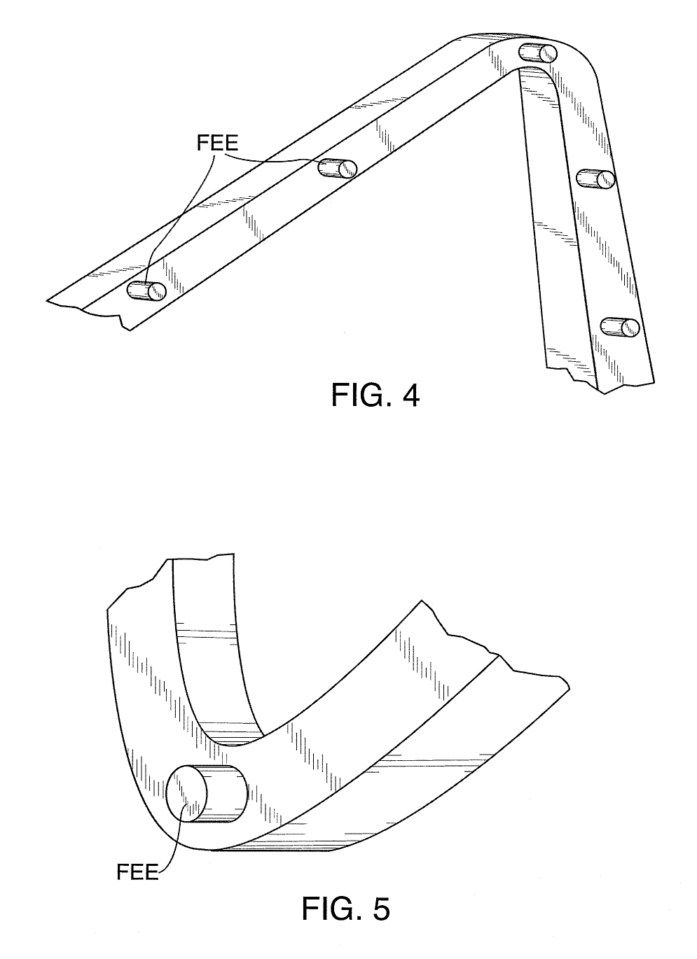

FIG. 4 illustrates a perspective view of a series of FEEs spaced along length of a strut section of a stent device.

FIG. 5 illustrates a detailed view of a cylindrically shaped FEE placed at the apex of a strut section of the stent device.

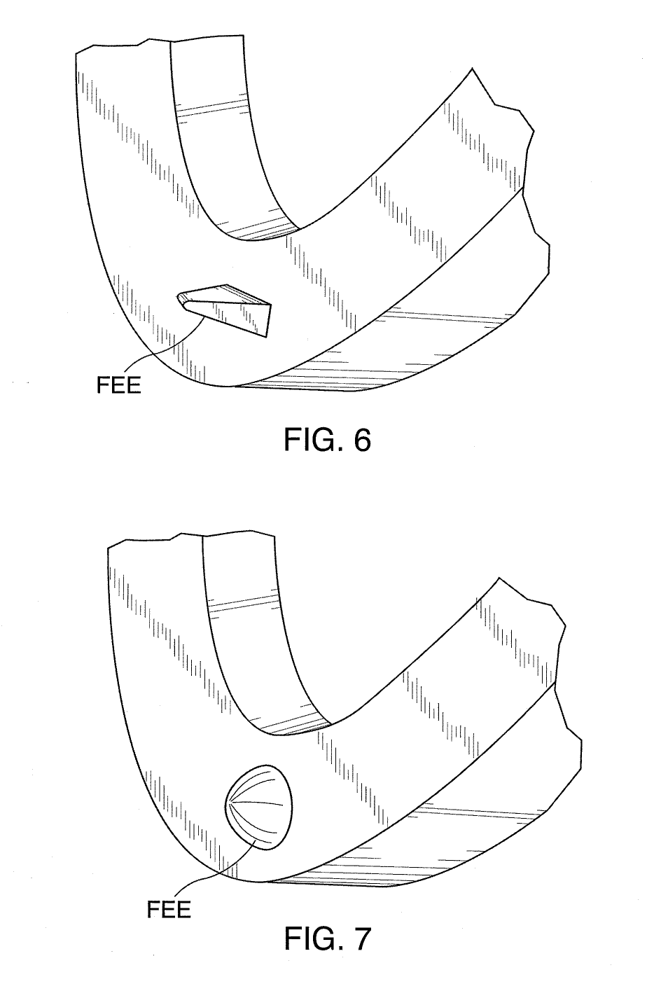

FIG. 6 illustrates a perspective view of a FEE formed as a pyramidal element at the apex of a strut section.

FIG. 7 illustrates a perspective view of a FEE formed as a dome element at the apex of a strut section.

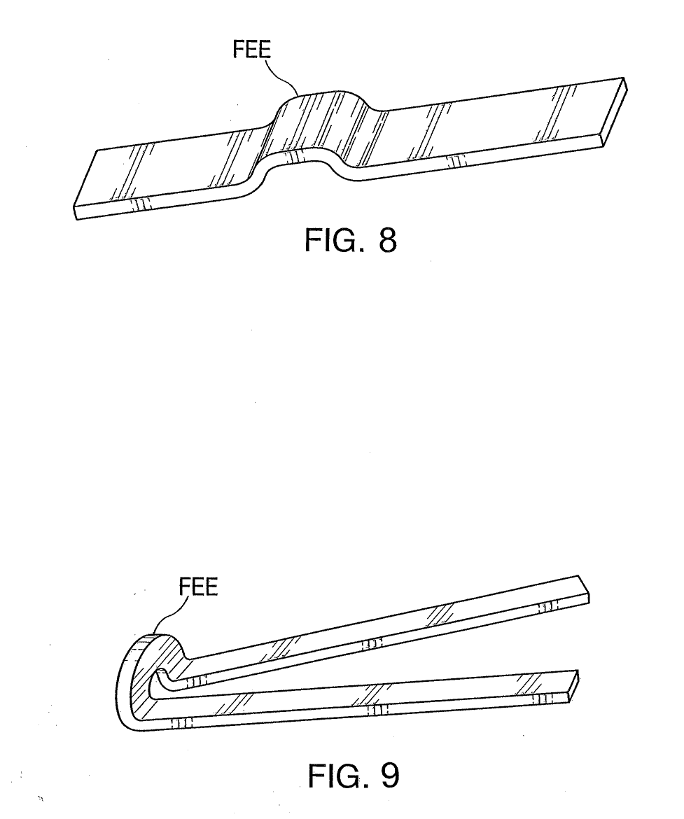

FIG. 8 illustrates a perspective view of a FEE formed by bending a portion of a strut length to raise it in height above the surface of the neighboring strut length.

FIG. 9 illustrates a perspective view of a FEE formed by bending the apex of a strut section upward.

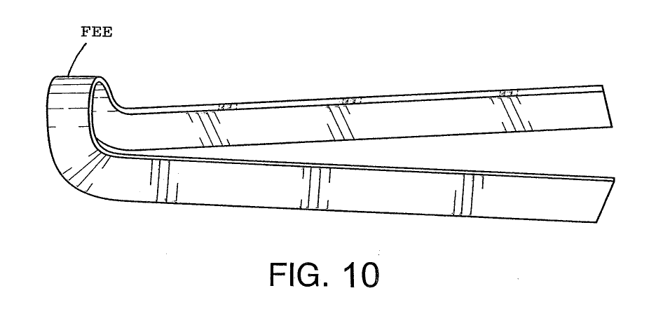

FIG. 10 illustrates a perspective view of a FEE formed by folding the apex of a strut section upward.

The FEE elements result in lifting away the majority of the stent structure of foreign material out of contact with body tissues that can induce or contribute to inflammation of tissue cells. When used in a blood vessel, the FEEs greatly reduce the surface area of the overall stent that can make contact with the blood vessel walls. This relief from contact also offers the ability of the blood vessel to move in a less constrained manner without generating friction with the stent.

The types of stent devices that can derive value from the inclusion of FEE's include devices that are introduced in the organ lumen using catheter based technology and devices that do not use catheters for placement in the body. Independent of the device being enhanced with FEEs, the objective is reduction in cellular response through a reduction of RMI and microfriction with the cellular wall. For example, catheter-delivered devices are designed to be expanded once placed in an organ lumen with the catheter. The design of stents typically includes strut beams that enable a range of folding for placement in a catheter and expansion upon deployment. The amount of outward force exerted against the organ lumen walls upon deployment, the alignment of stent struts to the organ cells, and the amount of bending or movement of the organ all influence the histological response of the body tissues to the foreign device. The FEEs can be strategically positioned on the stent struts to produce an effective reduction of metal contact with the lumen. As a mismatch of stent structure with cell orientation can produce microfriction generated by motion of the cells relative to the device, the FEEs reduction of contact area against the organ lumen walls will reduce the effect of elastic mismatch.

A stent device can be designed for optimal shaping and placement of FEE elements using the following metrics. A stent can be designed with FEEs for an optimally reduced Effective Metal Interface (EMI) with organ lumen walls. EMI_{F} represents a stent's Effective Metal Interface with the inclusion of FEE features:

$${EMI}_{F} = \frac{C ( 1 + {( n - {n}_{F} )}^{2} )}{{\sum }_{S = 1}^{x} {( l w - {l}_{F} {w}_{F} )}_{S}}$$

The effect of reducing the numerator by the number of strut intersections or bridges (n) that are lifted by the FEEs (n_{F}) and reducing the denominator for the surface area of struts (length l×width w) that are not in contact with the vessel wall by the FEE contact area l_{F}w_{F} produces a lower EMI that is a minor fraction of the EMI for a stent with no FEEs. It can be seen that the greatest effect on lowering EMI is the inclusion of FEEs at strut sections thereby reducing the numerator in the equation.

A metric for percentage change in outer diameter of the stent device is calculated below. The calculation is derived from the outer diameter of the stent design with no FEE and the artificial outer diameter of the stent with FEE where the outer diameter at the FEE points is measured as if the FEE where not compressible and the height of the FEE is an additive vector to the diameter at each FEE location. The percentage difference D_{H} in outer diameters is calculated as

$${D}_{H} = \frac{{D}_{FEE} - {D}_{Stent}}{{D}_{Stent}} \times 100 ( percent )$$

Preferably, the FEE features are designed so that D_{H} has a range of 4% - 0.1%.

Another metric for defining preferred FEE features is the FEE height to strut thickness ratio STR_{H}. FEEs require enough height such that they offer the function of lifting neighboring strut member high enough to make an overall impact on the EMI. The following equation illustrates this result.

$${STR}_{H} = \frac{{h}_{FEE}}{{t}_{S}}$$

The height h_{FEE} is measured from the outer diameter Dₛₜₑₙₜ of the stent without FEEs and represents the vector length of the FEEs oriented radially. The strut thickness is is measured at the location of the FEE. The h_{FEE} is in a preferred range of 0.02 mm to 4 mm, and most preferably in the range of 0.05 mm to 0.2 mm. The STR_{H} is preferably in the range of 0.05 to 3, and most preferably in the range of 0.2 to 0.7.

In a particularly preferred embodiment of the improved stent device, focal elevating elements on the stent structure are formed by laser-cutting or etching a cylindrical blank that has two outer-diameter levels of structure. For example, the cylindrical blank can have a stent mesh structure of a lesser outer diameter, and annular rings superimposed on the stent mesh structure having a greater outer diameter. The annular rings of greater outer diameter can then be laser-cut or etched to form spaced arrays of focal elevating is elements on the strut sections of the stent mesh structure.

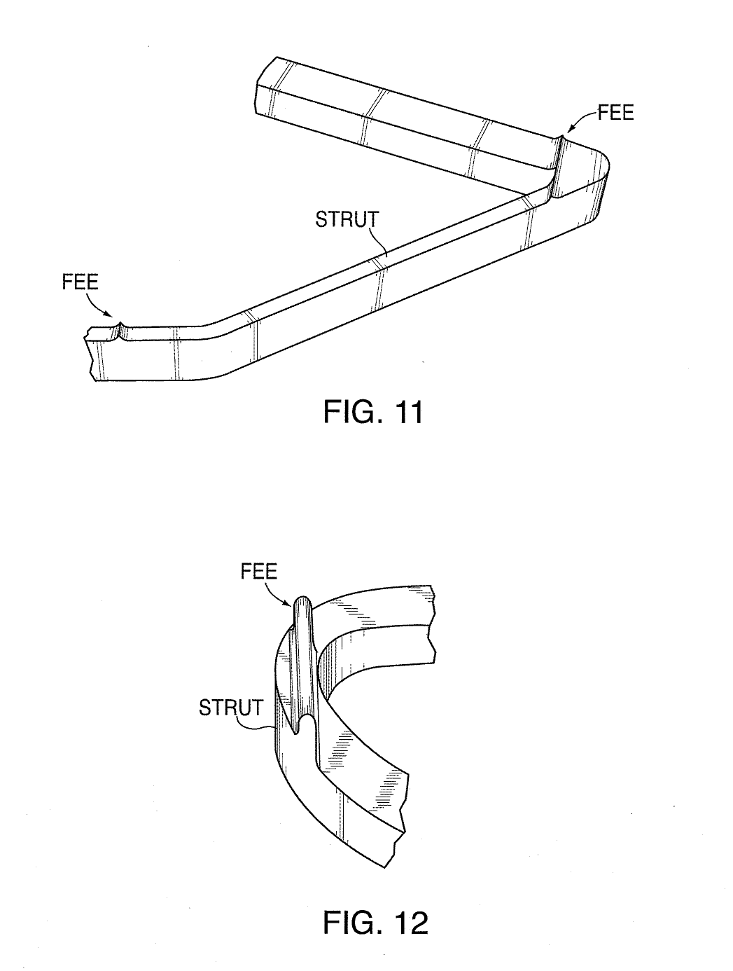

FIG. 11 illustrates a perspective view of focal elevating elements (FEEs) laser-cut or etched on the upward facing surface on the length or bend of a strut section of a stent mesh structure. The FEEs are shown as edged or fluke-like segments, which are more efficiently formed by laser-cutting or etching, rather than sharp points. However, when viewed on a macro-scale, the FEEs function like focal points.

FIG. 12 illustrates a perspective view of a FEE laser-cut or etched as a bar element on the bend of a strut section.

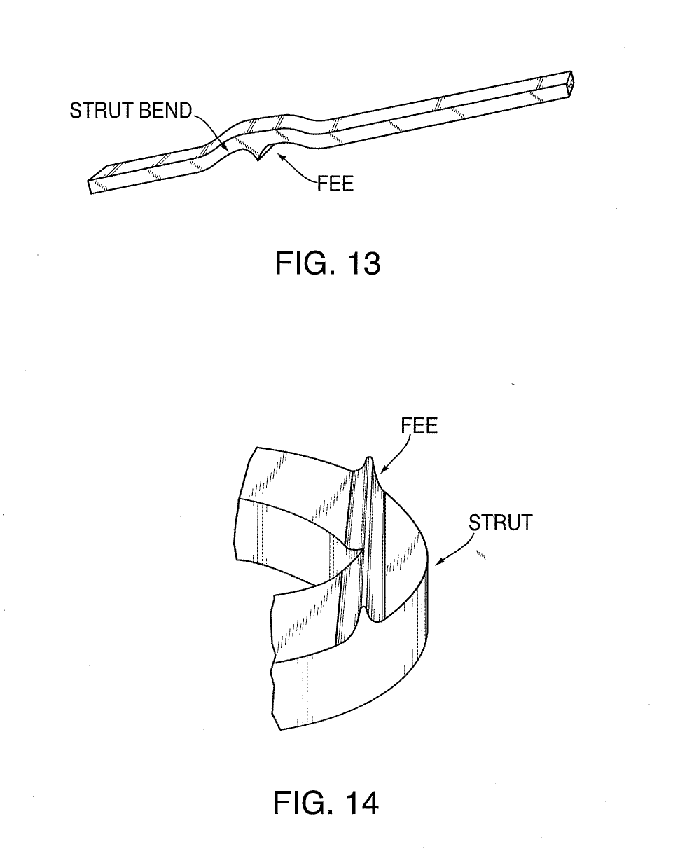

FIG. 13 illustrates a perspective view of a FEE laser-cut or etched as a fluke-like segment on a horizontal bend in the length of a strut section.

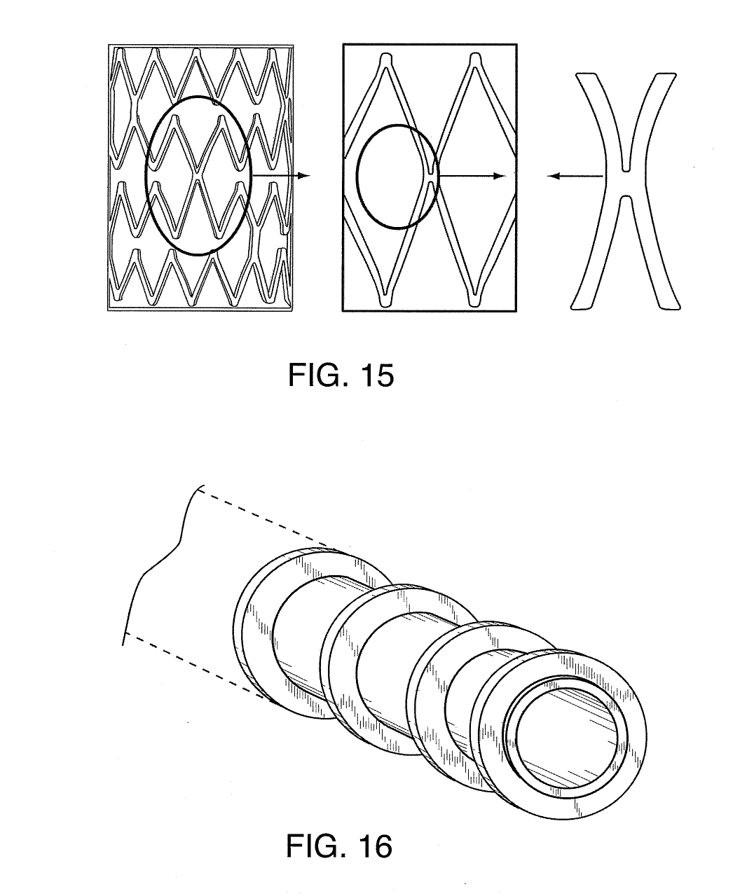

FIG. 14 illustrates a perspective view of a FEE laser-cut or etched as a fluke-like segment straddling a bend of a strut section.

A metric for defining a preferred FEE design for the fluke-like segments a ratio STR_{L} of the FEE segment length to strut thickness tₛ at the base of the FEE segment:

$${STR}_{L} = \frac{{l}_{FEE}}{{t}_{S}}$$

The length, l_{FEE}, is measured along the axial distance of the fluke-like FEE segment at the region where the FEE maintains the same height h_{FEE}. The l_{FEE} is preferably in the range of 0.01 mm to 0.5 mm. The l_{FEE} is shorter when the orientation of the strut where the FEE is located is circumferential or nearly circumferentially oriented, and can be longer in longitudinal orientations. The preferred range for STR_{L} is 0.2 to 10.

The number and location of FEEs affects the overall Effective Metal Interface (EMI). Experimental evidence indicates that the locations of the FEEs play a role in device behavior, stability, and cellular response. FEEs placed at bridges between circumferentially oriented strut rings, and at apexes of struts offer the majority of arterial injury relief. A pivotal design factor for reducing fatigue and arterial rubbing is to minimize the tendency of the overall structure to move as a unit. The design feature that contributes to this tendency is the bridges or strut linkages between concentric rings of stent wire mesh or strut sections. The concentric rings by themselves undergo the most micro movement during arterial extension/contraction or flexion. Since these elements see the majority of movement, the higher percentage of fatigue can be seen at bridges linking the concentric rings. By lifting the bridges through incorporation of the FEEs between concentric ring struts, the FEEs offer a series of relief points for the device. To further reduce the fatigue loading for a stent's concentric rings, the FEEs are preferably located at the apices of the stent struts. Examples of FEE features applied to concentric rings of stent struts at the bridges and apices of the stent struts are illustrated in the following figures.

FIG. 15 shows a sequence of progressively enlarged views of a stent mesh structure with rows of strut sections in the left-side view, two strut sections joining at a bridge portion in the center view, and a more detailed view of a bridge portion between adjacent strut sections in the right-side view. The bridge portions are the preferred locations for forming the focal elevating elements to minimize surface contact area between the stent and lumen walls.

FIG. 16 illustrates a cylindrical blank having two outer-diameter levels of structure for forming focal elevating elements by laser-cutting or etching. The wire mesh or strut sections of the stent body structure are not illustrated to provide a clearer view of the laser-cut or etched forming of focal elevating elements on concentric rings of stent strut bridges and apices.

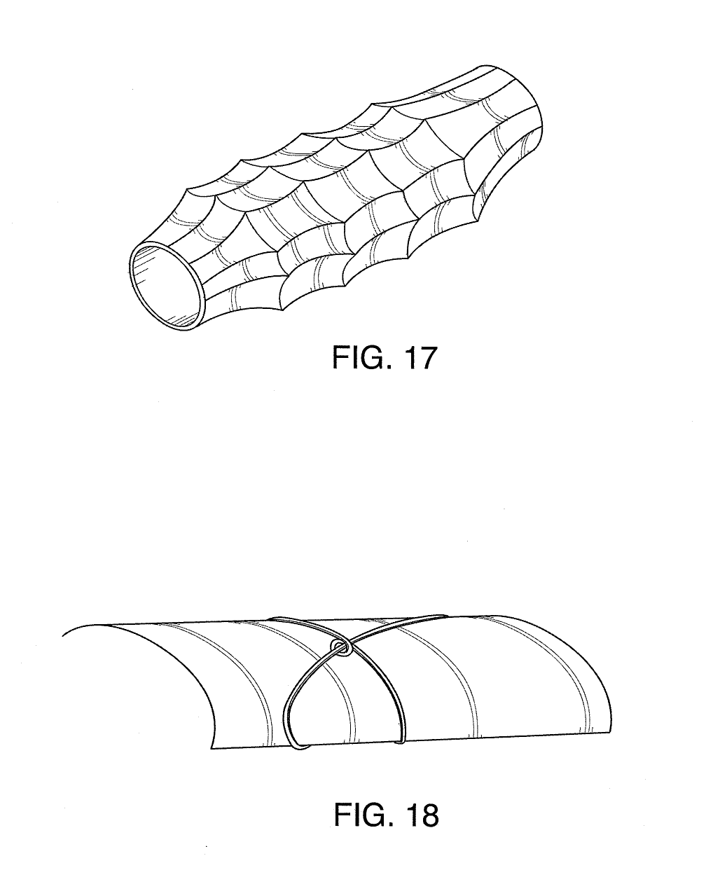

FIG. 17 illustrates the resulting stent structure's focal elevating elements by laser-cutting or etching the cylindrical tube in FIG. 16.

The fluke-like FEEs illustrated above are considered to be particularly effective in lifting key sections of the stent structural matrix, distributing pressure and maintaining stable contact with the lumen walls, while reducing the likelihood of the stent to migrate. Sharp-edged flukes may or may not penetrate slightly into the endolumenal cellular wall but whether or not they penetrate the wall the flukes offer stability while minimizing the number of cells adjoining the fluke head and anchoring the device to the placement location.

The following illustrates the extent of reduction in surface contact area of the fluke-like FEE segments compared to a rectangular strut section without FEEs. In the first equation, w is the width of a strut, l is the length of the strut, and A is the surface area of strut contact with the lumen walls without FEEs:

A=lw,

With the inclusion of FEEs, the height h of the FEEs will dictate the length of lumen walls free of contact. In the second equation, w_{F} is the width of a FEE, l_{F} is the length of the FEE, and LA is the area free of strut contact with the lumen walls:

LA=lw−2l_{F}w_{F}, where

sin θ=h/l_{F}

Other designs and modifications may be made in accordance with desired functional characteristics or stent properties. Outer points on the surface of the stent can be formed as focal elevating elements to embed into or press against plaque. An array of focal elevating elements can be used for linking annular bands of a stent with a plaque mass or blood vessel wall. The outer points can be made of a sufficiently rigid material to sustain a locking or engaging relationship with the blood vessel tissue and/or to pierce or engage the plaque and maintain the locking or engaging relationship therewith. The outer points can be designed to project at an angle of 90 degrees to the tangent of the annular band, or an acute angle may also be used. Further examples of designs for focal elevating elements are shown in the following figures.

FIG. 18 illustrates a perspective view of a FEE formed by twisting a loop at an intersection of wires of a stent mesh structure.

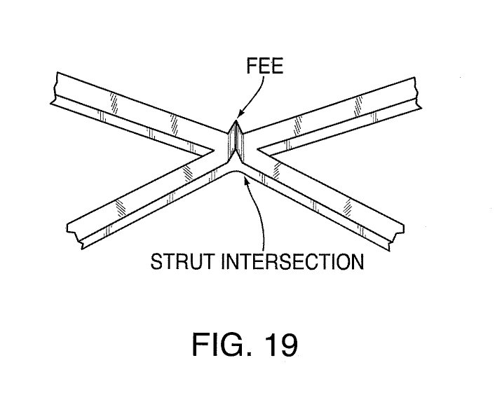

FIG. 19 illustrates a perspective view of a FEE laser-cut or etched as a fluke-like segment straddling the intersection of two crossing strut sections.

The focal elevating elements on the improved stent device may be formed as cylindrical, rectangular, spherical, conical, tear-drop, pyramidal, knife edge or inclined elements on the annular periphery of the stent device. They can be formed by bending or stamping a section of the stent structure, by an additive process (such as by welding, sputtering, or annealing on a peripheral surface), by a subtractive process (such as by grinding, oblation, or etching away surrounding material so that the bump element is higher than the surrounding surface, or by modifying small sections of the peripheral surface to be higher than the surrounding surface before or after sheet or tube cutting. For example, one method of modification of small sections of a mesh stent structure is by knotting, twisting, bending or weaving small sections of the wire mesh to produce raised elements from the mesh surface which are the interface with the artery wall of the stent devices.

Properly oriented and symmetrically positioned focal elevating elements can provide foci for expansion force. As the device exerts outward forces and the artery exerts inward forces, the focal elevating elements can be positioned at strategically located positions reducing the outward pressure of strut sections adjacent the focal elevating element.

The number and locations of focal elevating elements can affect the overall Relative Metal Surface Area (RMS) explained previously. They may be positioned along the lengths of the stent device surfaces such that a minimal amount of metal surface area is in contact with the artery wall. Focal elevating elements placed at bridges between circumferential strut rings or at the apices of strut sections of the stent device can offer a majority of arterial injury relief. When focal elevating elements are placed only at apices and bridges of strut members, the RMS of the strut members making up the concentric rings changes a little, while the RMS of the bridge portions is reduced more significantly due to its narrow length, thereby offering relief of relative motion of the circumferentially oriented strut rings.

The improved stent device with FEEs contain less structure and present contact points formed at minimal bridges and strut rings, thereby producing a small Effective Metal Interface (EMI). Stents with FEEs offer minimal histological response, and reduce the tendency of cellular injury and inflammation as compared to stents without FEEs.

It is to be understood that many modifications and variations may be devised given the above description of the principles of the invention. It is intended that all such modifications and variations be considered as within the spirit and scope of this invention, as defined in the following claims.

1. A stent, comprising:
a tubular stent body having a length and an outer expanded diameter sized to dimensions of an organ lumen in which it is to be used, and comprising a plurality of struts;
a plurality of focal elevating elements arranged in an array at spaced locations over the outer surface of the tubular stent body, said focal elevating elements having a height which results in an elevated outer diameter greater than the outer diameter of the strut sections of said stent body, and having a total contact surface area for contact with walls of the organ lumen that is a fraction of a total contact surface area of the outer surfaces of the strut sections of said tubular stent body, and being configured to apply point pressure to the organ lumen walls so as to elevate adjacent outer surfaces of the tubular stent body away from contact with the organ lumen walls;
wherein the stent body has at least a first thickness measured in a radial direction at a first point on a strut and a second thickness measured in a radial direction at a second point on a focal elevating element, and the second thickness is greater than the first thickness.

2. An improved stent device according to claim 1, wherein said focal elevating elements have regionally lifted sections that are configured to reduce pressure in regions neighboring the focal elevating elements in order to minimize trauma due to contact or movement between the device and the organ lumen walls.

3. An improved stent device according to claim 1, wherein said focal elevating elements are configured to reduce effective metal interface (EMI) of the stent device by minimizing overall material contact with the organ lumen walls.

4. An improved stent device according to claim 3, wherein EMI_{F} represents the Effective Metal Interface of said focal elevating elements of the stent device according to the following relation:
$${EMI}_{F} = \frac{C ( 1 + {( n - {n}_{F} )}^{2} )}{{\sum }_{S = 1}^{x} {( l w - {l}_{F} {w}_{F} )}_{S}}$$
in which n is the number of strut intersections of the stent device, while the number n_{F} are strut intersections that are lifted by said focal elevating elements, l is the length and w is the width for the surface area of each strut section, and l_{F} is the length and w_{F} is the width for the surface area of each focal elevating element, and
wherein EMI_{F} is a small fraction of the Effective Metal Interface for the improved stent device compared to the EMI of a stent without focal elevating elements.

5. An improved stent device according to claim 1, wherein D_{H} is a percentage difference in outer diameters of said focal elevating elements compared to said stent body structure:
$${D}_{H} = \frac{{D}_{FEE} - {D}_{Stent}}{{D}_{Stent}} \times 100 ( percent ) ,$$
and
wherein D_{H} is preferably in a range of 4%-0.1%.

6. An improved stent device according to claim 1, wherein STR_{H} is a ratio of the height h_{FEE} of said focal elevating elements compared to thickness tₛ of the strut sections of said stent body structure:
$${STR}_{H} = \frac{{h}_{FEE}}{{t}_{S}} ,$$
and
wherein STR_{H} is preferably in a range of 0.05 to 3, and most preferably in the range of 0.2 to 0.7.

7. An improved stent device according to claim 6, wherein h_{FEE} is in a preferred range of 0.02 mm to 4 mm, and most preferably in the range of 0.05 mm to 0.2 mm.

8. An improved stent device according to claim 1, wherein said focal elevating elements are formed on strut sections of a stent body structure in a configuration selected from the group consisting of: fluke shaped and spaced along a length of a strut sections including the strut intersections; fluke shaped and placed at apices of strut sections of the stent device; pyramidal shaped and placed along the length, at strut intersections and at apexes of strut sections; dome shaped and placed along the length, at strut intersections and at apexes of strut sections; bent or stamped from a portion of a strut to raise it in height above the surface of neighboring strut lengths; apex of a strut section bent upward; and apex of a strut section folded upward.

9. An improved stent device according to claim 1, wherein said focal elevating elements are formed by laser-cutting or etching a cylindrical blank that has two outer-diameter levels of structure: a cylindrical blank for said stent body structure having a lesser is outer diameter; and annular concentric rings superimposed on the stent body structure having a greater outer diameter, wherein the annular rings of greater outer diameter are laser-cut or etched to form spaced arrays of focal elevating elements on the stent body structure.

10. An improved stent device according to claim 1, wherein said focal elevating elements are small fluke-like segments of a given length formed on strut sections of said stent body structure.

11. An improved stent device according to claim 10, wherein STR_{L} is a ratio of length l_{FEE} of the fluke-like segments extending at the height of said focal elevating elements to strut thickness tₛ at the base of the segments:
$${STR}_{L} = \frac{{l}_{FEE}}{{t}_{S}} ,$$
and
wherein STR_{L} is in a preferred range of 0.2 to 10.

12. An improved stent device according to claim 10, wherein the small fluke-like segments for said focal elevating elements are formed by laser-cutting, grinding or etching a cylindrical blank that has two outer-diameter levels of structure: a cylindrical blank for said stent body structure having a lesser outer diameter; and annular concentric rings superimposed on the stent body structure having a greater outer diameter, wherein the annular rings of greater outer diameter are laser-cut, ground or etched to form spaced arrays of fluke-like segments on the stent body structure.

13. An improved stent device according to claim 10, wherein the fluke-like segments are oriented to extend along a longitudinal axis or perpendicular axis of the stent device or at an angle oriented longitudinal or perpendicular to the endoluminal axis.

14. An improved stent device according to claim 1, wherein said focal elevating elements are formed on bridge portions joining adjacent annular rows of strut sections.

15. An improved stent device according to claim 1, wherein said focal elevating elements are formed on adjoining apices of adjacent annular rows of strut sections.

16. An improved stent device according to claim 1, wherein said focal elevating elements are each formed as a twisted loop of wire at an intersection of wire mesh sections of said stent body structure.

17. An improved stent device according to claim 1, wherein said focal elevating elements are each formed as a fluke-like segment straddling an intersection of crossing strut sections of said stent body structure.

18. An improved stent device according to claim 1, adapted for use in a blood vessel lumen.

19. An improved stent device according to claim 18, adapted for use to retain plaque dissection in a blood vessel lumen after balloon angioplasty or other endovascular intervention.

20. An improved stent device according to claim 1, adapted for use as a scaffold in a tubular body structure of the group consisting of: arteries, veins, bile ducts, pancreatic ducts, ureters, trachea, bronchus, ear canal, eye canal, sinuses, intestinal tracts, and tubular organ structures.

21. An intraluminal prosthesis, comprising:
a stent body configured to scaffold a lumen in which it is deployed, and comprising a plurality of strut sections;
a plurality of radially outwardly protruding elements disposed at spaced apart locations and extending from the outer surface of the stent body, said radially outwardly protruding elements protruding, in an unconstrained state ex-vivo, to an outside radius that is greater than the outside radius of strut sections adjacent the radial protruding elements, the radial protruding elements having a total contact surface area for contact with walls of the lumen that is a fraction of a total contact surface area of the outer surfaces of the stent body, and the radial protruding elements configured to produce an outwardly directed force gradient in-vivo that is greatest at the protruding element and decreases at locations spaced away from the protruding elements, so to minimize pressure being applied against the lumen walls at locations spaced apart from the radially protruding elements

22. The intraluminal prosthesis of claim 21, wherein the radially protruding elements protrude by a radial dimension that is sufficient to produce a gap between portions of the outer surface of the stent body and a tubular body within which the stent prosthesis is constrained, the gap being greatest adjacent to the radially protruding element and decreasing at locations spaced farther therefrom.

23. The intraluminal prosthesis of claim 21, wherein the radially protruding elements comprise point contact zones.

24. The intraluminal prosthesis of claim 23, wherein the radially protruding elements are arranged in an array along the length of the stent body.

25. The intraluminal prosthesis of claim 21, wherein the radially protruding elements comprise elongate contact segments oriented in a direction corresponding to the direction of movement of the tissue to be scaffolded.
